(19) United States
(12) Patent Application Publication
(10) Pub. No.: US 2025/0176994 A1
(43) Pub. Date: Jun. 5, 2025

(54) APPARATUS AND METHOD TO GUIDE THE INSERTION OF MEDICAL DEVICE INTO A SUBJECT

(71) Applicant: SONONURSE VS INC., Verdun (CA)

(72) Inventor: Yahia ZINE, Sainte-Agathe-des-Monts (CA)

(21) Appl. No.: 18/844,560

(22) PCT Filed: Mar. 8, 2023
(86) PCT No.: PCT/CA2023/050306

§ 371 Date: Sep. 6, 2024

Related U.S. Application Data
(60) Provisional application No. 63/375,134, filed on Sep. 9, 2022.
(60) Provisional application No. 63/268,986, filed on Mar. 8, 2022.

(51) Int. Cl. A61B 17/34; A61B 8/00

(52) U.S. Cl. CPC A61B 17/3403; A61B 8/4218; A61B 8/4281; A61B 8/4422; A61B 2017/3413

(57) ABSTRACT

An apparatus to guide movement of an insertable medical device is provided. The apparatus comprises a single plane articulated arm adapted to be affixed to an ultrasound transducer and configured to allow the adjustment of a medical device within a single displacement plane, the displacement plane being coplanar with a scanning plane of the ultrasound transducer. A multi-plane ultrasound device comprising a plurality of transducers to simultaneously feed images of multiples planes.

### CROSS-REFERENCE TO RELATED APPLICATIONS

The present patent application claims the benefits of priority of U.S. Provisional Patent Application No. 63/268,986, entitled “APPARATUS AND METHOD TO GUIDE MOVEMENT OF AN INSERTABLE MEDICAL DEVICE”, and filed at the United States Patent and Trademark Office on Mar. 8, 2022 and the benefits of priority of U.S. Provisional Patent Application No. 63/375,134, entitled “APPARATUS AND METHOD TO GUIDE THE INSERTION OF MEDICAL DEVICE INTO A SUBJECT”, and filed at the United States Patent and Trademark Office on Sep. 9, 2022, the contents of which are incorporated herein by reference.

### FIELD OF THE INVENTION

The present invention generally relates to apparatuses and methods to guide a movement of medical devices inserted in a patient or subject. More particularly, the present invention generally relates to apparatuses and methods using ultrasound transducers to guide the insertion of medical devices into a patient.

### BACKGROUND OF THE INVENTION

In the field of medical in-plane needle alignment, medical practitioners typically use ultrasound transducers to insert a needle within a vein or artery of a patient. The practitioner typically aligns the needle within the scanning plane of the ultrasound transducer so that the output image of the transducer shows the entire shaft and tip of the needle. In doing so, medical practitioners typically rely on their experience and intuition to operate the necessary equipment by freehand. As such, only a few practitioners have enough skills and experience to position the needle appropriately within the necessary plane. Indeed, the practitioner may easily lose track of the needle in the output image upon a slight movement of the transducer as the ultrasound beam is thin. Understandably, existing techniques require a high level of expertise as well as excellent hand-eye coordination, and only capable of being performed by highly trained specialists.

Existing guides configured to align a needle with the scanning plane of a transducer are attached to the transducer. The transducer further comprises a small hole which acts as a guide for a needle. However, these systems are typically configured to only receive a needle and don't allow the installation of a venous catheter or other larger medical devices. They are also disposable and therefore costly. In other instances, such systems have to be disinfected prior to each usage.

Moreover, in anesthesiology, ultrasound is commonly used to guide the placement of needles when injecting local anesthetic solutions in the proximity of nerves identified within an ultrasound image. It is also used for vascular access such as cannulation of large central veins and for difficult arterial cannulation. Prior art ultrasound transducers provide medical practitioners a view of a single scanning plane-typically a cross-sectional or longitudinal view of the needle (as shown in FIGS. 1 and 2, respectively). However, neither of these single scanning planes provides all the information necessary to perform such medical procedures. For example, the ultrasound guided application of an anesthetic requires the placement of a needle in close proximity of a nerve without penetrating it. The use of a single scanning plane may therefore provide a medical practitioner information regarding the placement of the needle relative to the periphery of the nerve or the depth of the needle, but not both.

There is therefore a need for an apparatus adapted to position and maintain a needle within the image plane of an ultrasound transducer while allowing the necessary range of motion. There is further a need for an ultrasound transducer or transducer mount capable of selectively providing a view of multiple scanning planes.

### SUMMARY OF THE INVENTION

The shortcomings of the prior art are generally mitigated by an apparatus to guide movement of a medical device. In an aspect of the invention, an articulated arm adapted to be affixed to an ultrasound transducer and configured to allow the adjustment of a medical device within a single displacement plane is provided. The displacement plane allowed by the arm is coplanar with a scanning plane of the ultrasound transducer.

In another aspect of the invention, an ultrasound transducer mount is provided. The ultrasound transducer is configured for the ultrasound transducer to selectively pivot about its longitudinal axis to provide a range of longitudinal views of a desired vessel or nerve.

In the present document, the views of a vein may interchangeably refer to viewing nerves, arteries or any other target body portion of a subject or patient. In another aspect of the invention, a dual plane ultrasound transducer comprising a plurality of transducers is provided.

In a further aspect of the invention, an apparatus to guide movement of a medical device insertable in a physical body is provided. The apparatus comprises a mounting assembly for receiving an ultrasound transducer, the ultrasound transducer mount being rotatable around the receivable ultrasound transducer, an articulated arm having a first end attachable to the mounting assembly and a second end attachable to the insertable medical device, the articulated arm moving along a single plane to position a longitudinal axis of the medical device in-plane.

The articulated arm may be used to rotate the mounting assembly. The mounting assembly may comprise a pivoting base for receiving the ultrasound transducer and a rotation mechanism engaging rotation of the pivoting base, which may have a shape substantially matching a shape of a bottom portion of the ultrasound transducer. The pivoting base may comprise a suspending mechanism adapted to receive a bottom portion of the ultrasound transducer. The pivoting base may further comprise a liquid fillable sealed chamber between a bottom portion of the ultrasound transducer and a body portion of the subject. The pivoting base may be shaped to lock with a bottom portion of the ultrasound transducer. The pivoting base may comprise a brake to maintain the pivoted base to a specific angle.

The second end of the articulated arm may be selectively attachable and detachable from the insertable medical device, and may comprise a medical device attaching assembly for mounting yet unmounting the insertable medical device using one hand. The medical device attaching assembly may comprise a touchless cover removal mechanism to remove a cover of the insertable medical device. The touchless cover removal mechanism may comprise a slidable member to move the cover of insertable medical device away of the insertable medical device. The slideable member may be attached to the medical device using an aseptic no touch technique (ANTT). The medical device attaching assembly may be a slideable chariot. The medical device attaching assembly may comprise a lateral force absorption member. The lateral force absorption member may be resilient along a longitudinal axis of the medical device. The second end of the articulated arm may also comprise a slidable member to move the insertable medical device toward and away of the mounting assembly, which may conform with an external surface of the medical device, and may be integral with the ultrasound transducer.

In yet another aspect of the invention, a method to guide movement of an insertable medical device is provided. The method comprises holding a mounting assembly adapted to receive an ultrasound transducer using a first hand, attaching the insertable medical device to the mounting assembly using a second hand, rotating the mounting assembly about an axis substantially normal to the received ultrasound transducer using the second hand, fixing the rotated mounting assembly to a desired angle, inserting the medical device in-plane in a target area of the patient by moving the attached insertable medical device using the second hand.

The method may further comprise releasing the medical device using the second hand, removing a cover of the insertable medical device prior to inserting the medical device without touching the said cover, and pulling and pushing the medical device to test movement of the said medical device.

In yet another aspect of the invention, a gel dispenser for an ultrasound transducer is provided. The gel dispenser comprises a top portion attachable to the ultrasound transducer and a bottom portion for contacting a body part of a user, the bottom portion comprising a peripheral wall and central concave portion in-between the wall to lower pressure on tissues in-plane. The peripheral wall may comprise pillars at corners of the bottom portion.

In yet another aspect of the invention, an assembly for attaching yet detaching a medical device insertable in a physical body to an ultrasound transducer using a single hand is provided. The assembly comprises a member for releasably attaching the medical device to the assembly to be in-plane, a member for pivotably attaching the assembly to an elongated arm of the ultrasound transducer.

The assembly may further comprise a touchless cover removal mechanism to remove a cover of the insertable medical device. Said touchless cover removal mechanism may comprise a slideable member to move the cover of insertable medical device toward and away of the insertable medical device. Said assembly may be a slideable chariot and may further comprise a lateral force absorption member, and said medical device may be pre-attached to the assembly.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is an exploded perspective view of an embodiment of an apparatus to guide the insertion of a medical device into a subject according to the principles of the present invention.

FIG. 2 is a perspective view of the apparatus of FIG. 1.

FIG. 3 is a perspective view of an attachment mechanism of a medical device attachable to an elongated arm of an apparatus to guide the insertion of a medical device into a subject according to the principles of the present invention, the medical device being shown unclipped.

FIG. 4 is a perspective view of an attachment mechanism of a medical device attachable to an elongated arm of an apparatus to guide the insertion of a medical device into a subject according to the principles of the present invention, the medical device being shown clipped.

FIG. 5 is a bottom perspective view of an embodiment of an apparatus to guide the insertion of a medical device into a subject shown with a detached base.

FIG. 6 is a bottom perspective view of an articulated arm of the apparatus of FIG. 5 shown with a base mounted thereto.

FIG. 7 is a perspective view of the apparatus of FIG. 5 shown with the medical device detached from the articulated arm.

FIG. 8 is a perspective view of the apparatus of FIG. 7 with the medical device attached to the articulated arm.

FIG. 9 is a rear perspective view the apparatus of FIG. 8 shown with the attached medical device being pivoted.

FIG. 10 is a side perspective view of the front of the apparatus of FIG. 9.

FIG. 11 is a side perspective view of the apparatus of FIG. 10 shown with a cover of the medical device being removed.

FIG. 12 is a side perspective view of the apparatus of FIG. 11 shown with the cap of the medical device, removed.

FIG. 13 is a perspective view of the apparatus of FIG. 12 shown with the medical device prior to be inserted into a body portion of a subject.

FIG. 14 is a perspective view of the apparatus of FIG. 13 as the medical device is being inserted into a body portion of a subject.

FIG. 15 is a perspective view of the apparatus of FIG. 14 with the medical device being detached from the attachment mechanism.

FIG. 16 is a perspective view of the apparatus of FIG. 15 with the medical device being detached from the elongated arm.

FIG. 17 is perspective view of a catheter detached from the apparatus of FIG. 16 shown while being removed from the needle.

FIG. 18 is perspective view of the catheter of FIG. 17 being completely removed from the needle.

FIG. 19 is a perspective view of another embodiment of an apparatus to guide the insertion of a medical device into a subject according to the principles of the present invention.

FIG. 20 is a perspective view of the apparatus of FIG. 19 shown with a transducing device being detached from a pivoting base

FIG. 21 is a perspective view of another embodiment of an apparatus to guide the insertion of a medical device into a subject according to the principles of the present invention comprising a slidable member shown with the medical device pushed toward the apparatus.

FIG. 22 is a perspective view of the apparatus of FIG shown with the medical device pulled away from the apparatus.

FIGS. 23 to 25 are a side views of an embodiment of an apparatus to guide the insertion of a medical device into a subject according to the principles of the present invention shown in different positions, during positioning, prior to insertion and during insertion, respectively.

FIG. 26 is a top perspective view of yet another embodiment of an ultrasound mount holding an ultrasound device in accordance with the principles of the present invention

FIG. 27 is a side elevation view of the ultrasound mount of FIG. 26.

FIG. 28 is a front perspective view of the ultrasound mount of FIG. 26.

FIG. 29 is a bottom perspective view of the ultrasound mount of FIG. 26.

FIG. 30 is a back perspective view of the ultrasound mount of FIG. 26.

FIG. 31 is another bottom perspective view of the ultrasound mount of FIG. 18.

FIG. 32 is a perspective view of an embodiment of an attachment assembly for a medical device in accordance with the principles of the present invention.

FIG. 33 is side elevation view of the attachment assembly of FIG. 32.

FIG. 34 is side elevation view of the attachment assembly of FIG. 32 shown being pushed toward the cover.

FIG. 35 is a perspective view of the attachment assembly of FIG. 32 shown with the slidable member being pushed.

FIG. 36 is a perspective view of an embodiment of a rotation assembly of a transducer in accordance with the principles of the present invention.

FIG. 37 is a perspective view of another embodiment of a rotation assembly of a transducer in accordance with the principles of the present invention.

FIG. 38 is a perspective view of yet another embodiment of a rotation assembly of a transducer in accordance with the principles of the present invention, the rotation assembly comprising a first embodiment of a locking mechanism.

FIG. 39 is a perspective view of yet another embodiment of a rotation assembly of a transducer in accordance with the principles of the present invention, the rotation assembly comprising a second embodiment of a locking mechanism.

FIG. 40 is a perspective view of the locking mechanism of FIG. 39 shown with a handle.

FIG. 41 is a bottom perspective view of the locking mechanism of FIG. 39 shown with a handle.

FIG. 42 is a top plan view of a locking member of FIG. 39 shown in a locked and unlocked positions.

FIG. 43 is a perspective view of an embodiment of a gel chamber of a transducer in accordance with the principles of the present invention.

FIG. 44 is a perspective view of another embodiment of a gel chamber of a transducer in accordance with the principles of the present invention.

FIG. 45 is a perspective view of an embodiment of a calibration device installed on a transducer in accordance with the principles of the present invention.

FIG. 46 is a perspective view of another embodiment of a calibration device installed on a transducer in accordance with the principles of the present invention.

FIG. 47 is perspective view an embodiment of an arm attachment assembly adapted to calibrate said arm of a transducer in accordance with the principles of the present invention, shown without the transducer.

FIG. 48 is perspective view the arm attachment of FIG. 47, shown with the transducer.

FIG. 49 shows a plan view and side view of yet another embodiment of an ultrasound mount in accordance with the principles of the present invention.

FIG. 50 is an illustration of an embodiment of a dual plane transducer in accordance with the principles of the present invention.

FIG. 51 is a top plan view of multiple embodiments of a multi plane transducer in accordance with the principles of the present invention.

FIG. 52 is a side view of an embodiment of an ultrasound mount holding an ultrasound device in accordance with the principles of the present invention.

### DETAILED DESCRIPTION OF THE INVENTION

An apparatus and method to guide movement of a medical device will be described hereinafter. Although the invention is described in terms of specific illustrative embodiments, it is to be understood that the embodiments described herein are by way of example only and that the scope of the invention is not intended to be limited thereby.

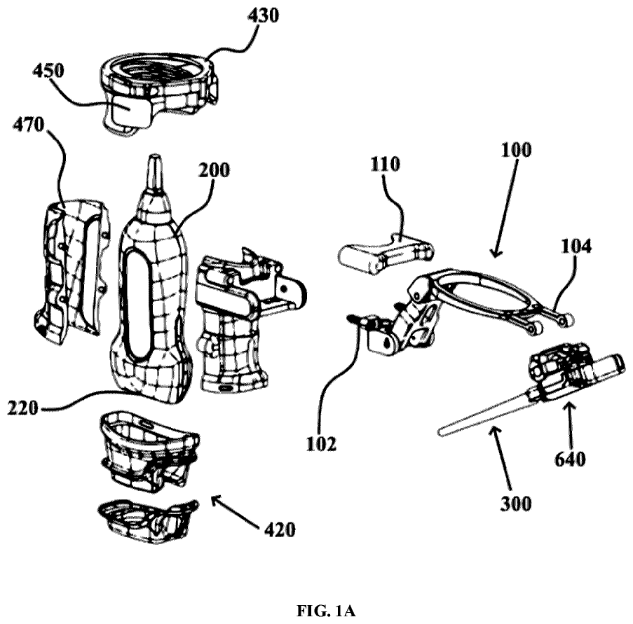

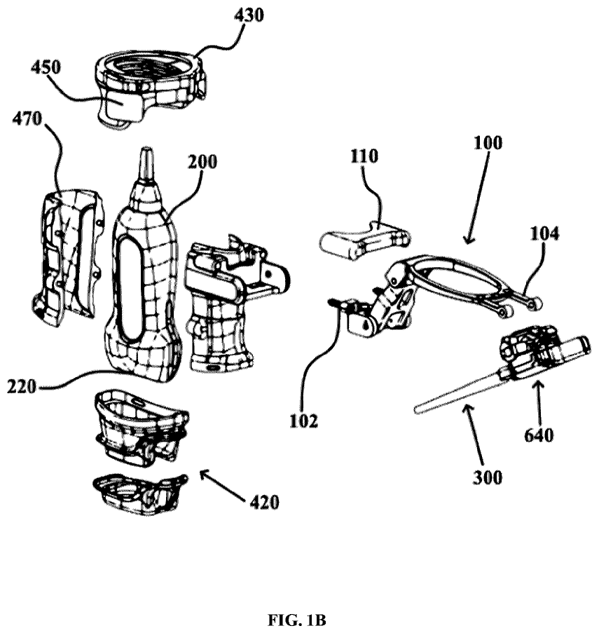

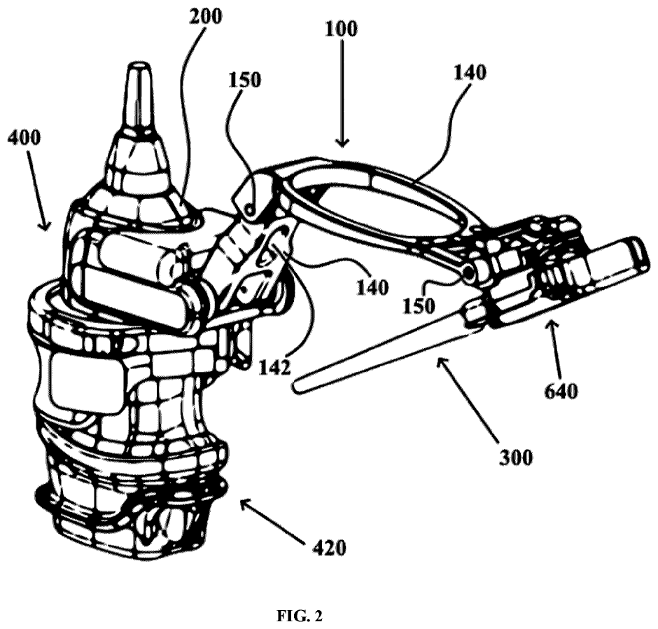

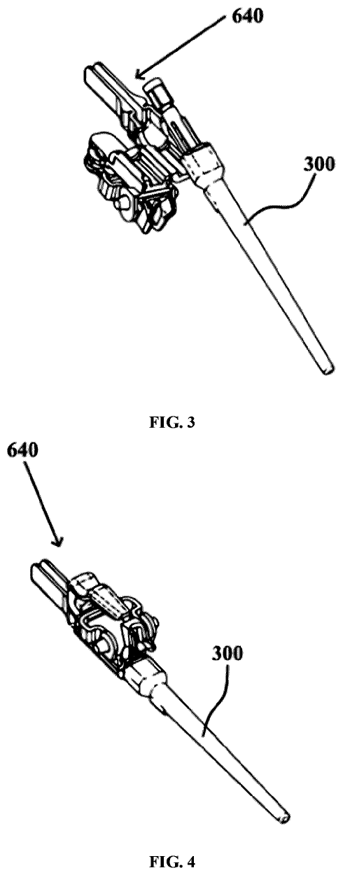

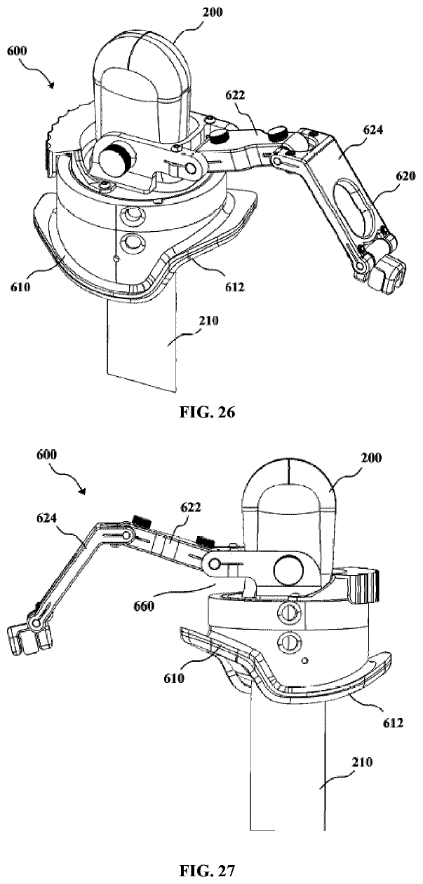

Referring to FIGS. 1 to 20, an embodiment of an ultrasound mount or apparatus to guide movement of a medical device is illustrated. In such an embodiment, the apparatus comprises an articulated arm 100 moving in a single plane. Broadly, the articulated arm 100 comprises a proximal end 102 adapted to be affixed to an ultrasound transducer 200, and a distal end 104 configured to receive a medical device 300 to be inserted into a patient. Notably, the articulated arm 100 is configured to guide a movement of the medical device 300 to be inserted. In some embodiments, the apparatus may comprise an attaching assembly 640 pivotally mounting the medical device 300. In some embodiments, the articulated arm 100 is adapted to position and maintain a longitudinal axis of the medical device 300 within a predetermined plane 210 (shown at FIG. 26) while allowing a desirable range of motion within said plane 210. In a typical embodiment, the plane 210 is substantially vertical or normal/orthogonal relative to a bottom portion 220 of the ultrasound transducer 200 in contact with the patient or with the skin of the patient. Such technique is known as inserting a medical device in-plane. Understandably, it is desirable that the articulated arm 100 be rotated to be substantially aligned to the scanning plane or image detected by the ultrasound transducer 200 to allow its operator to maintain visibility of the medical device 300 throughout the procedure.

The present invention may be configured to be used by medical practitioners or nurses in the injection or catheterization of superficial veins. Accordingly, the ultrasound transducer 200 may comprise a high frequency continuous ultrasound. In such embodiments, the ultrasound transducer may be used to detect veins at a depth of under 5 cm. The medical device 300 may be, but is not limited to, a needle, a catheter or any other medical device requiring ultrasound guidance during a procedure or insertion.

In certain embodiments, as illustrated in FIG. 2, the articulated arm 100 may comprise a plurality of arms or sections 140 having any suitable length, longitudinal shape and/or cross-sectional shape. The articulated arm 100 may further comprise one or more joints 150 interconnecting two or more adjoining sections 140. The joints 150 may be bolted joints, prismatic joints or any other joints suitable for allowing relative movement between two adjoining sections 140 within a single plane. In a preferred embodiment, the articulated arm 100 comprises a suitable number of sections 140 and joints 150 to enable a desirable range of motion of the medical device 300. In the illustrated embodiment, the combination of joints 150 and sections 140 allows lowering the medical device 300 toward the patient while providing a translation movement along the predetermined plane. As such, and as an example, the medical device 300 may be longitudinally inserted and withdrawn in a vein, artery or any other target or portion of a patient.

To avoid contamination, the articulated arm 100 may be operated entirely by manipulation of the arms 140 and/or joints 150, and without necessitating a manipulation of the medical device 300.

In some embodiment, the articulated arm 100 may further be configured to be operated by a robot or other automated system. In such embodiments, one or more of the sections 140 and/or joints 150 may comprise attachment points (not shown) configured to be connected to one or more actuators of the robot. Alternatively, the joints 150 may be actuated or motorized to operate one or more of the sections 140. Understandably, this may further encourage asepsis of the procedure.

In certain embodiments, one or more of the sections 140 and/or joints 150 may be made of a sterilizable material to allow said components to be sterilized and reused for multiple medical procedures. In other embodiments, one or more of the sections 140 and/or joints 150 may be pre-sterilized and disposable for a single use procedure. In other embodiments still, the articulated arm 100 and the ultrasound transducer 200 may be configured to be wrapped in a protective sheath to be used in a medical or surgical procedure with greater surgical asepsis.

As previously stated, the articulated arm 100 is configured to be affixed to the ultrasound transducer 200 in a position to allow the articulated arm to be maneuvered within the predetermined plane 210. In certain embodiments, the articulated arm 100 may be removably affixed to the ultrasound transducer 200 thereby allowing an operator to selectively attach or remove the articulated arm 100 as desired. To that end, the proximal end 102 of the articulated arm 100 may comprise a fastening means 110 adapted to secure yet detach the articulated arm 100 to the ultrasound transducer 200. The fastening means 110 may be configured to secure the articulated arm 100 to the ultrasound transducer 200 in a predetermined position or, alternatively, the ultrasound transducer 200 may comprise a fixation point 230 adapted to receive the fastening means. In a further embodiment, the articulated arm 100 may be attached to the handle 210 of the ultrasound transducer 200. As such, the attachment of the articulated arm 100 to the handle 210 may allow pivoting or rotational movement around said handle 210 to allow movement of the articulated arm 100 in a different substantially vertical plane or orthogonal to the bottom portion 220 of the transducer 200.

In certain embodiments, the articulated arm 100 may further comprise a fastening joint 190 at its proximal end 102. The fastening joint 190 may be fastened to the fastening means or may form part thereof. The fastening joint 190 may be configured to allow a limited and/or predetermined range of motion of the articulated arm 100. Understandably, in other embodiments, fastening joints 190 may allow different ranges of motion of the articulated arm 100. For example, fastening joint 190 may define a circular or arcuate range of motion of the articulated arm 100 and medical device 300 along a single predefined plane.

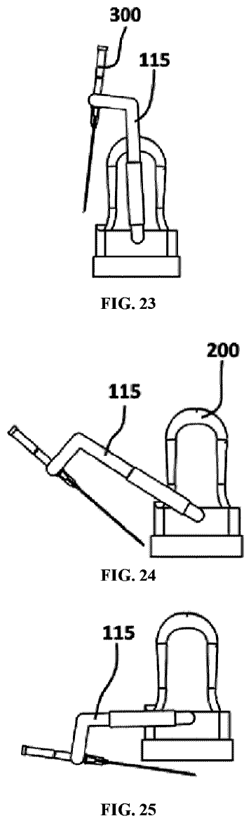

In yet other embodiments, the dimensions and arrangement of the sections 140 may allow the articulated arm 100 to fold into a retracted configuration (see for instance FIGS. 2 and 23) when not in use. The ultrasound transducer 200 may further comprise a recess 142 or compartment configured to receive the articulated arm 100 in said retracted configuration to prevent it from interfering with a procedure when not in use.

In certain embodiments, the installation of the articulated arm 100 on the ultrasound transducer 200 may be performed by a specialized technician to ensure that the position of the medical device 300 is at all times within the scanning plan of the ultrasound transducer 200. The installation of the articulated arm 100 may require laser alignment tools to ensure that the medical device 300 is always displaced within the scanning plane of the ultrasound transducer 200 with a possible deviation of up to 0.1 mm. It may be appreciated that by limiting the range of motion of the articulated arm 100 and medical device 300, a medical practitioner may locate the medical device 300 within the scanning plane of the ultrasound transducer 200 with greater ease.

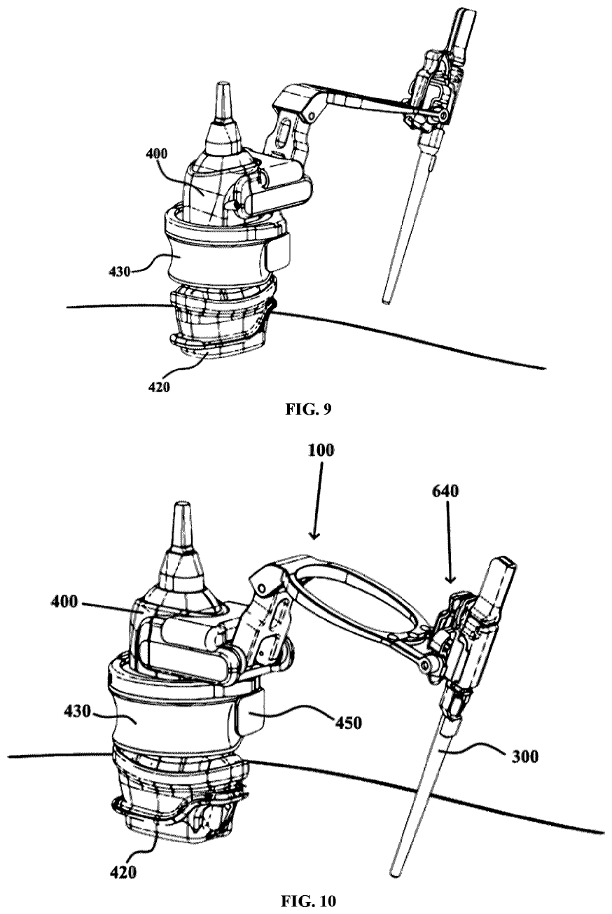

In further embodiments, the distal end 104 of the articulated arm 100 may comprise an attachment means or connector (not shown) adapted to receive and be secured to an additional section 140. To that end, the articulated arm 100 may be extended to allow an additional range of motion of the medical device 300. In some embodiments, the articulated arm 100 may comprise a plurality of attachments points for receiving the medical device 300. Referring now to FIGS. 9 and 10, an exemplary attachment point or means 160 is illustrated. In some embodiments, the attachment point 160 comprises a support 162 configured to receive the medical device 300 and an actuator 164 adapted to operate the medical device 300. In certain embodiments, the support 162 may further comprise grooves (not shown) adapted to receive a portion of the medical device 300 and thereby limit its range of motion.

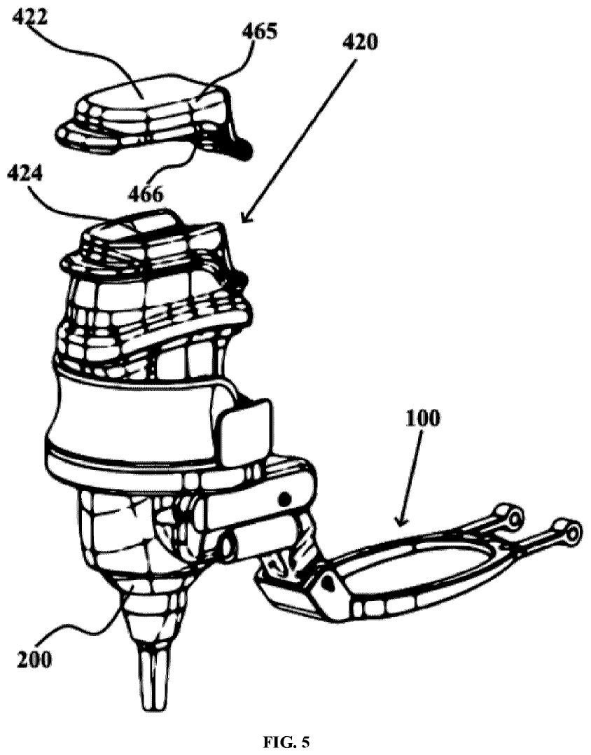

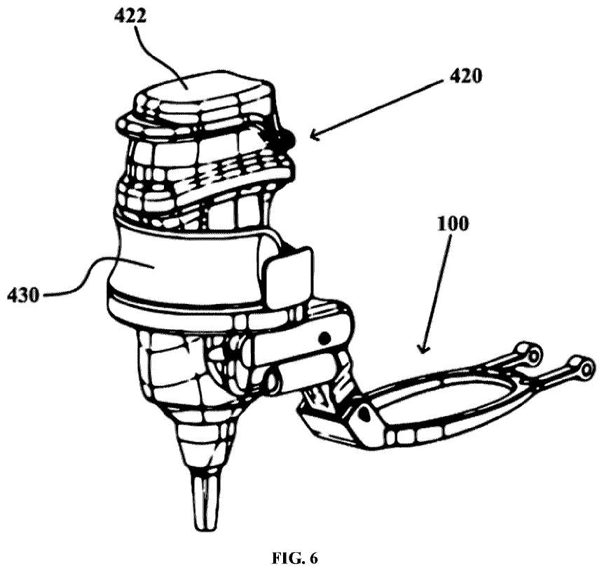

Referring now to FIGS. 5 to 17, a method to guide movement of an insertable medical device into a subject is shown. Referring to FIGS. 5 and 6, the device comprises a gel dispenser 422 and 424 disposing a lubricant or gel on the skin of the subject during operations of the ultrasound transducer.

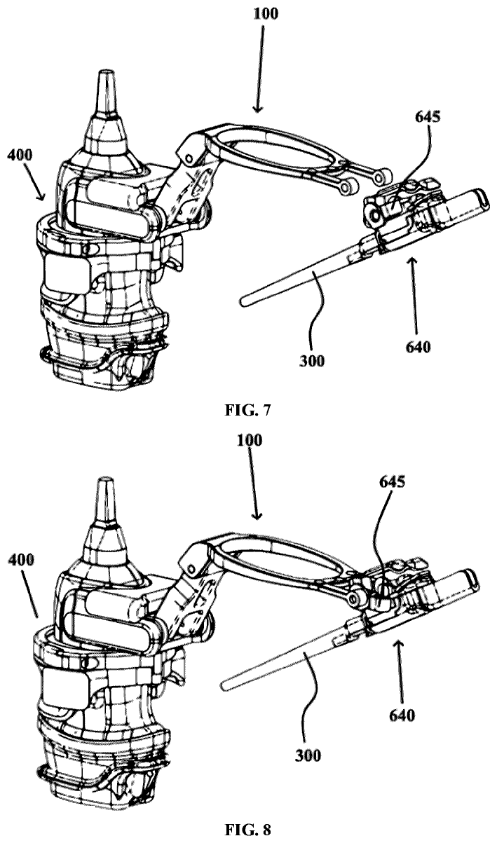

Referring now to FIGS. 7 and 8, the method further comprises attaching the medical device to the elongated arm 100 of the apparatus. In some embodiments, the apparatus may comprise an attachment assembly 640 comprising an attachment means to attach yet detach the medical device 300. The attachment means may be embodied as a locking mechanism 645 allowing the operator to secure the attaching assembly 640 to the articulated arm 100 using only one hand.

Referring now to FIG. 9, once the medical device 300 is pivotally attached to the articulated arm 100, the method further comprises the operator, holding the apparatus or transducer using a first hand, rotating the mount around the transducer using a second hand, typically the dominant hand of the operator. The rotation is stopped when the operator detects an inner body part of interest of the subject using the transducer. The method may further comprise locking the articulated arm 100 at the set angle using the first hand of the operator (holding the transducer or mount).

Referring now to FIGS. 10 and 11, the method may further comprise removing a cover or protective cap of the medical device using the second hand of the operator. In the illustrated embodiment, the operator presses a resilient button which release the cover. The method may further comprise pushing the cap 315 away from the medical device still using the second hand. In the illustrated embodiment, the pushing is performed by sliding a chariot or member toward the cover still using the second hand. The push let the cover move away from the remainder of the medical device.

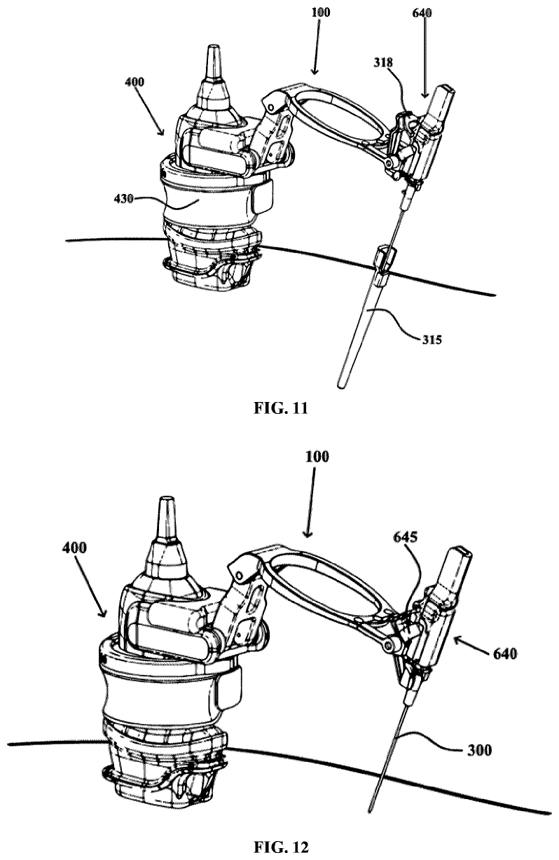

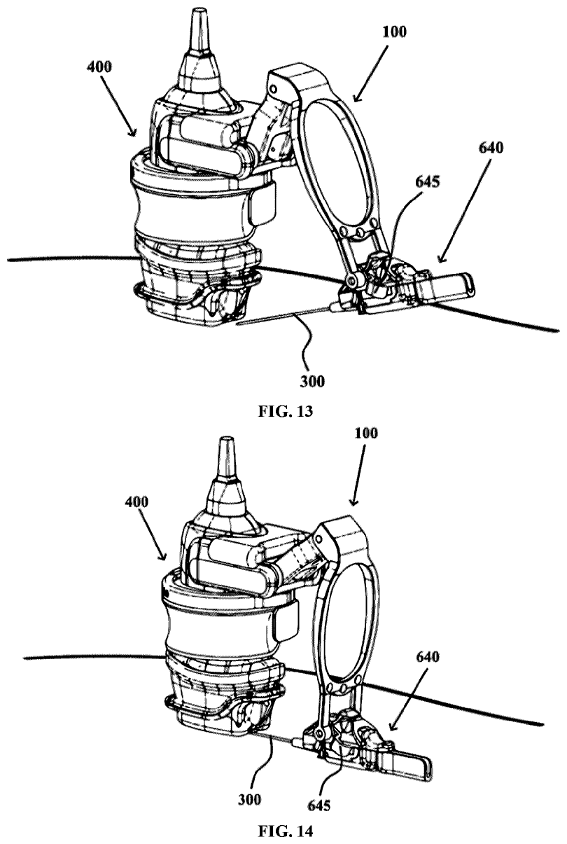

Referring to FIGS. 12 to 13, the method may further comprise manipulating the elongated arm to move the medical device within a single plane or in-plane corresponding to the plane detected by the transducer. The operator moves the medical device toward the skin of the subject to insert the said medical device in-plane. Understandably, the articulated arm 100 is constrained in the said angle during manipulation of the medical device to ensure that the medical device is inserted in-plane.

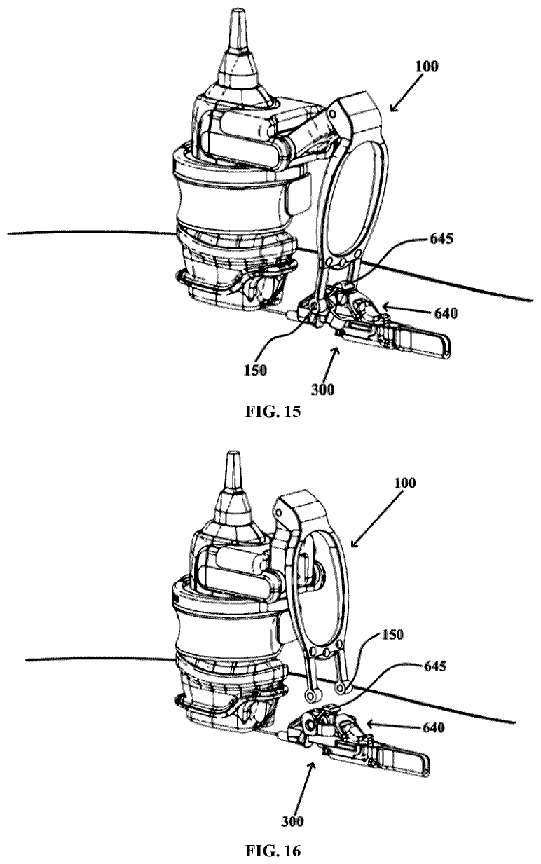

Referring now to FIGS. 14 and 15, when the medical device is inserted in the skin of the subject, the method may further comprise detaching a portion of the inserted medical device, such as a catheter. As such, the operator may then manipulate the detached portion of the medical device without being limited by the elongated arm 100.

Referring now to FIG. 16, the method may further comprise detaching the medical device or attachment mechanism from the elongated arm 100. In the illustrated embodiment, the attachment mechanism comprises a resilient member pushing joint pins inward to release the medical device or attachment mechanism from an end of the elongated arm 100.

Referring now to FIGS. 17 and 18, a step to remove a catheter from the medical device is shown. The method may further comprise using a member 317 to provide side movement to the medical device during operations.

Referring to FIGS. 1 to 18, the apparatus may for example be used to carry out a procedure using an aseptic no touch technique (ANTT). The first hand of the user holds, navigates and maintains the ultrasound transducer 200 during the entire procedure. The second hand of the user, which is preferably the dominant hand of the user, rotates the articulated arm 100, installs the medical device 300, removes the protective cap 315 of the medical device 300, punctures the skin of the patient, and releases the medical device 300 from the articulated arm 100. Thereafter, the dominant hand may then be used to remove the ultrasound transducer 200. Thus, the apparatus allows to perform the entire procedure with one hand on the ultrasound transducer 200 and the other on the articulated arm 100, without having to directly touch the medical device 300 and thus avoid contamination. In a preferred embodiment, the operator manipulates the medical device 300 with his/her dominant hand while holding the transducer 200 with his/her other hand.

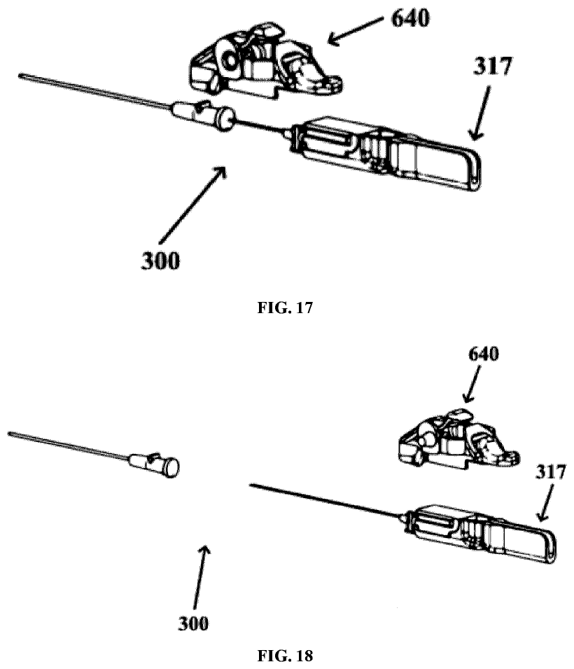

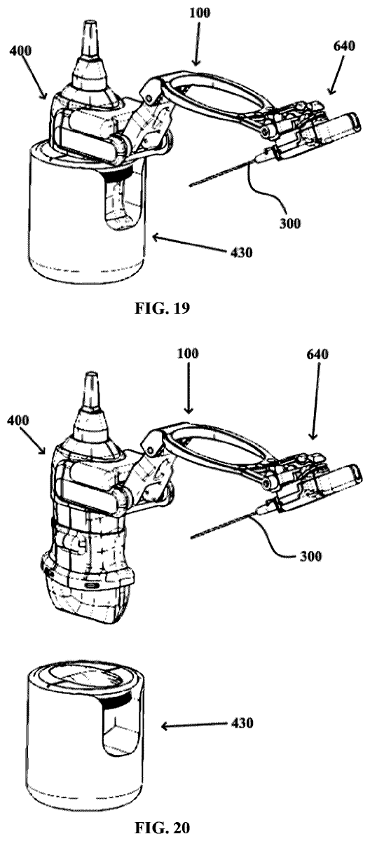

Referring now to FIGS. 19 and 20, another embodiment of an apparatus to guide insertion of a medical device is illustrated. The apparatus comprises a mount 400 attached to an ultrasound transducer. The apparatus further comprises base to be pressed against the skin of the subject. The base comprises a rotative mount adapted to receive and rotate a bottom portion of the transducer. The base 430 comprises a sealed bottom portion allowing passage of rays. As such, the bottom portion 435 of the base 430 may be made of plastic or other material allowing passage of rays. The bottom portion of the base may be partially filed with a liquid such as water. The liquid generally aims at reducing or limiting the bubbles which may affect quality of the detected plane. FIG. 19 illustrated the transducer being rotatably be mounted on the base 430 and FIG. 20 illustrated the transducer being unmounted from the rotative base 430.

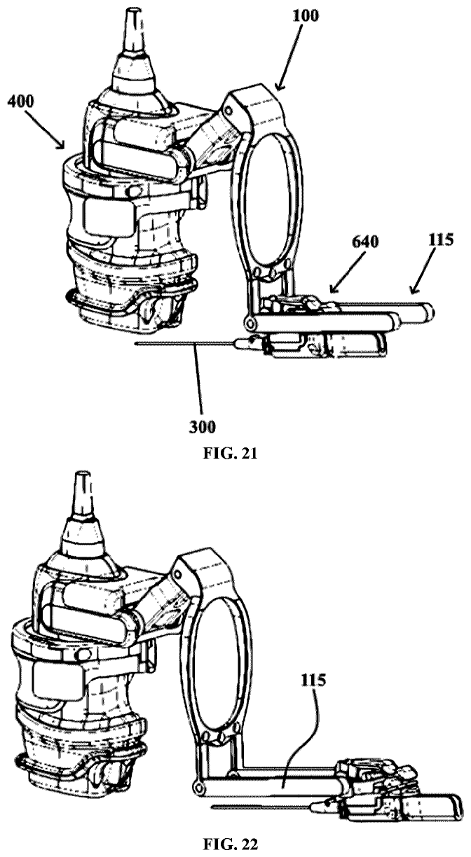

Referring now to FIGS. 21 and 22, an embodiment of the attachment mechanism 640 comprising a slidable member 115 is illustrated. The slidable member 115 generally allows the medical device to move toward or away the subject or transducer. As such, an operator may insert the medical device into a subject by sliding. The slidable member 115 may comprise a longitudinal groove receiving a matching protrusion of the attachment mechanism 640 or of the medical device 300.

Referring now to FIGS. 23 to 25, another embodiment of the apparatus to guide an insertable medical device in as subject is shown. In such embodiment, the articulated arm 100 comprises a slidable member or section 115. The slidable member 115 may be adapted to receive the attaching assembly 640, thus giving the articulated arm 100 a further translational degree of freedom which may be used, for example, to move or insert the medical device 300 into a body portion of the patient. FIG. 23 shows the slidable member 115 in an expended position with the medical device 300 away from the mount 400 as it is pivoted. FIG. 24, shows the slidable member 115 in an expended position while the articulated arm 100 is being pivoted. FIG. 25 show the slidable member 115 in compressed or retracted position while the medical device 300 is inserted in the subject.

The present invention is further directed to a method of guiding the medical device 300 in a medical procedure. The method may comprise fastening or attaching the articulated 100 to the ultrasound transducer 200. Fastening the articulated 100 to the ultrasound transducer 200 may comprise securing the proximal end 102 of the articulated arm 100 to the handle 210 of the ultrasound transducer 200. In certain embodiments, the method may further comprise aligning the articulated arm in relation to the ultrasound transducer 200. In some embodiments, the aligning may be performed using a laser.

The method may further comprise displacing the medical device 300 along the predetermined plane 10 of the articulated arm 100 to maintain said medical device 300 within the scanning plane of the ultrasound transducer 200. Displacing the medical device 300 may comprise selectively pivoting one or more of the sections 140 of the articulated arm 100 about the joints 150. In certain embodiments, the method may further comprise attaching or detaching further sections 140 to modify the range of motion of the medical device 300. In certain embodiments, selectively pivoting one or more of the sections 140 may comprise engaging robotic actuators.

The present invention is further directed to an ultrasound transducer mount 400 configured to retain the ultrasound transducer 200 and to allow its rotation about a rotation axis, preferably a longitudinal axis. Such mounting generally aims at varying the scanning plane of the ultrasound transducer 200 in operation to find the inserted medical device in the patient.

In some embodiments, such as the embodiment shown at FIGS. 1 and 52, the mount 400 may comprise a transducer attaching mechanism 410, such as but not limited to a strap or to matching portions. In such embodiment a pivoting base 420 is configured to receive a conventional ultrasound transducer 200, a rotation mechanism 430 configured to engage a rotation of the pivoting base 420, and one or more adjustment knobs 440 to selectively control said rotation mechanism 430. The rotation mechanism 430 may comprise a geared system (such as bevel gears) or any other suitable system.

In certain embodiments, the mount 400 may further comprise a locking mechanism configured to lock the rotation of the pivoting base 420 about its pivoting axis after a desirable pivot angle has been obtained.

In some embodiments, the mount 400 comprises two matching portions attachable to one another. As such, each portion covers a side of the transducer and, when attached together, conform with the contour of the external surface of the transducer (as shown in FIG. 1).

Referring to FIGS. 5 and 6, in certain embodiments, the mount may further comprise an ultrasound gel dispenser 422 and 424 fluidly connected to an ultrasound gel reservoir (not shown). The ultrasound gel dispenser 422 and 424 may be selectively or automatically actuated to dispense an ultrasound gel onto the surface of the patient's skin under the ultrasound device. As ultrasound sound waves have a difficult time traveling through the air, ultrasound gel may be used to reduce the gap between a patient and the ultrasound transducer 200 to reduce acoustic impedance and reflection thereby allowing a clearer image to be produced. The ultrasound gel may however impede or complicate the insertion of the medical device 300 in proximity of the ultrasound transducer 200. Accordingly, the mount 400 may additionally comprise a peripheral skirt 465 configured to scrape and collect excess ultrasound gel dispensed during operation of the ultrasound transducer 200. Understandably, the peripheral skirt 465 may facilitate the use of the articulated arm 100 and medical device 300 by partially or completely removing excess ultrasound gel. The peripheral skirt 465 may comprise walls to form a container-shape 466 allowing collection and retention of the said gel.

Referring now to FIGS. 5 and 6, in certain embodiments, the pivoting base 420 may comprise a silicon membrane base or gel dispenser 422, a gel base or any other suitable material adapted to allow soundwaves emitted by the ultrasound transducer 200 to pass through with little or no interference. The silicon membrane 424 or gel base may form a gap of about 2 to 7 mm between the ultrasound transducer 200 and the surface of the patient's skin. In certain embodiments, the silicon membrane or gel base may allow the ultrasound transducer 200 to provide a better or clearer ultrasound image of the internal body structure at a depth of under 1 cm. It may be appreciated that conventional ultrasound transducers provide a blurry image of the internal structure at shallow depths.

According to an exemplary use of the mount 400, a medical practitioner may detect a vein, an artery or any other target of a patient by displacing the pivoting base 420 and attached ultrasound transducer 200 across the surface of the patient's skin. Once a cross-sectional view of the desired vein, artery or other target is detected (typically appearing as a circular element on a display), the medical practitioner may tighten the strap of the mount 400 around the patient's member and rotate the pivoting base 420 about its axis by means of the rotation mechanism 430 and the one or more adjustment knobs. By rotating the pivoting base 420 about its fixed position relative to the targeted vein, the ultrasound transducer 200 may thus be automatically positioned to provide a longitudinal view of the targeted vein with little or no further adjustment by the medical practitioner.

In certain embodiments, the adjustment knobs may be manually actuated by a one or more robotic actuators (not shown). In certain embodiments, the robotic actuators may further be operated by an automated system. In certain embodiments, the one or more robotic actuators may be actuated and operated by an artificial intelligence system configured to identify a suitable rotation of the rotation mechanism 430 and ultrasound device 200 to provide a desired longitudinal view of a selected vein.

During medical procedures involving the use of the ultrasound transducer 200, medical practitioners and nurses may unwillingly apply excessive force onto the ultrasound transducer 200, the force being normal to the surface of the patient's skin. The excess force may apply an undesirable pressure onto the patient's member thereby reducing the cross-sectional area of a targeted vein rendering in less visible within the scanning plane. Still referring to FIG. 1, the mount 400 may therefore comprise a suspension assembly 470 disposed between the mount 400 and the ultrasound transducer 200. The suspension assembly 470 is generally configured to maintain a desirable level of pressure on the patient's member under examination. The suspension assembly 470 may comprise a spring, an air suspension assembly, a magnetic suspension assembly, or any other suitable system for the storage of mechanical energy. Understandably, the suspension assembly 470 may therefore prevent excess pressure between the ultrasound transducer 200 and the surface of the patient's skin. In yet other embodiments, the base of the transducer 200 may have an oval shape allowing the device 200 to follow the shape of the skin of the patient instead of having the skin of the subject being pulled by the device 200. The bottom portion may further be concave to limit the pressure level directly under the transducer 200.

In certain embodiments, the suspension assembly 470 may comprise vertical suspension members and horizontal suspension members. The vertical suspension members may reduce the pressure generated by a vertical force applied to the mount 400. The horizontal suspension members may further reduce unintentional horizontal vibrations of the ultrasound transducer 200 during a scanning procedure.

In embodiments having a peripheral skirt 465, it may be appreciated that the peripheral skirt 465 may further assist in reducing the undesirable pressure onto the patient's member by providing a larger surface area for the application of the force onto the patient's skin.

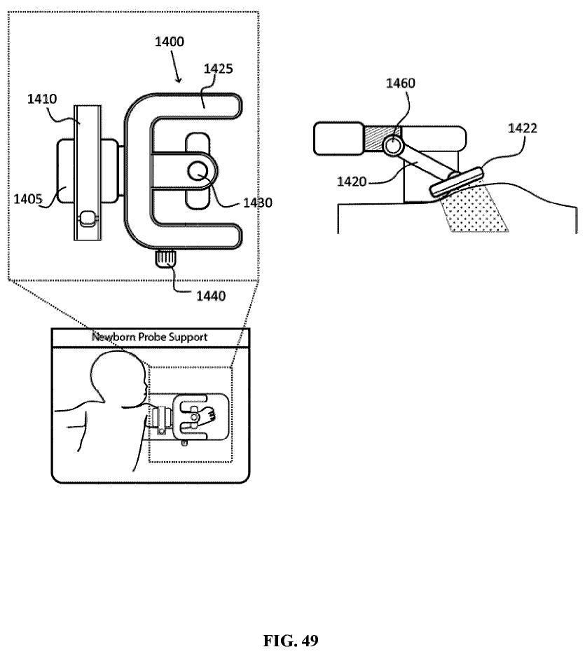

Referring now to FIG. 49 an ultrasound transducer mount 1400 according to another embodiment is illustrated. The mount 1400 comprises a support 1405, a strap 1410, a pivoting arm 1420 configured to receive an ultrasound transducer 1422, a rotation mechanism 1430 configured to engage a rotation of the ultrasound transducer 1422, one or more support arms 1425 and one or more adjustment knobs 1440 to selectively control said rotation mechanism 1430. The rotation mechanism 1430 may comprise a motor, a geared system or any other suitable drive system. In certain embodiments, the mount 1400 may similarly comprise a suspension assembly 1460 disposed between the support 1405 and the pivoting arm 1420 configured to maintain a desirable level of pressure on the patient's member under examination. The supporting arms 1425 may similarly assist in reducing an undesirable pressure onto the patient's member by providing a larger surface area for the application of the force onto the patient's skin.

During certain procedures, an arm of the patient may not be sufficiently large to be received within the strap 410, 1410. In such instances, the mount 400, 1400 may further comprise an arm support 480 configured to receive and retain the patient's arm while providing a larger surface for the strap 410, 1410 to be secured.

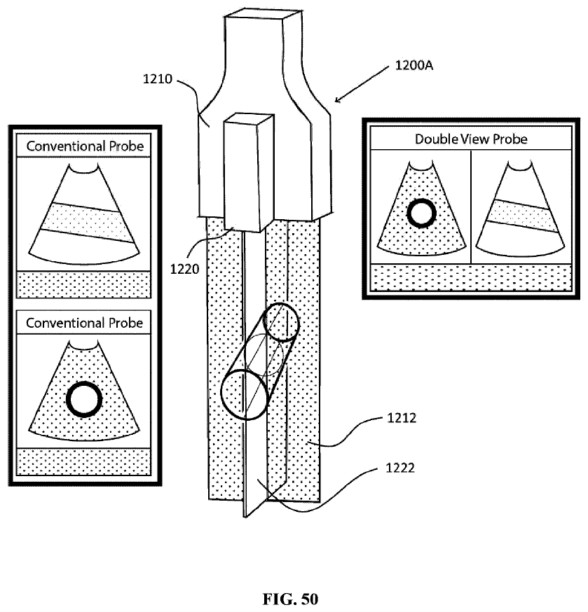

The present invention is further directed to a multi-plane ultrasound device 1200 comprising a plurality of transducers. In the embodiment illustrated in FIG. 50 the multi-plane ultrasound device 1200A comprises two transducers configured to provide two distinct scanning planes. Specifically, the dual plane ultrasound device 1200A comprises a primary transducer 1210 providing a first scanning plane 1212, and a secondary transducer 1220 providing a second scanning plane 1222. In certain embodiments, the secondary transducer 1220 is substantially orthogonal to the primary transducer 1210 along their longitudinal axes.

Still referring to FIG. 50, the multi-plane ultrasound device 1200A may comprise a casing or enclosure 1230 configured to protect the plurality of transducers 1200, such as the primary and secondary transducers 1210, 1220 and to define the relative positioning therebetween.

As previously stated, anesthesiology requires the ultrasound guidance of a needle in proximity of a nerve. Notably, the multi-plane ultrasound device 1200A may allow an injection to be made at a desirable proximity of the nerve without injecting directly into it. According to an exemplary use of the multi-plane ultrasound device 1200A, the primary and secondary transducers 1210, 1220 may provide respectively a cross-sectional and longitudinal view of the nerve.

In certain embodiments, the secondary transducer 1220 may have a narrower scanning plane than the primary transducer 1210. For example, the secondary transducer 1220 may provide a scanning plane having a lateral dimension of about 1 cm to 6 cm.

It may be appreciated that the multi-plane ultrasound device 1200A may continuously provide a medical practitioner a view of the medical device 300 during insertion. For example, the secondary transducer 1220 may provide a first view of the medical device as it's being directed to the target vein or nerve, while the primary transducer 1210 may provide a traditional cross-sectional view of said vein or nerve. The simultaneous views may thus reduce the risks involved in the insertion of a medical device and positioning of the said inserted medical device in the body of a patient.

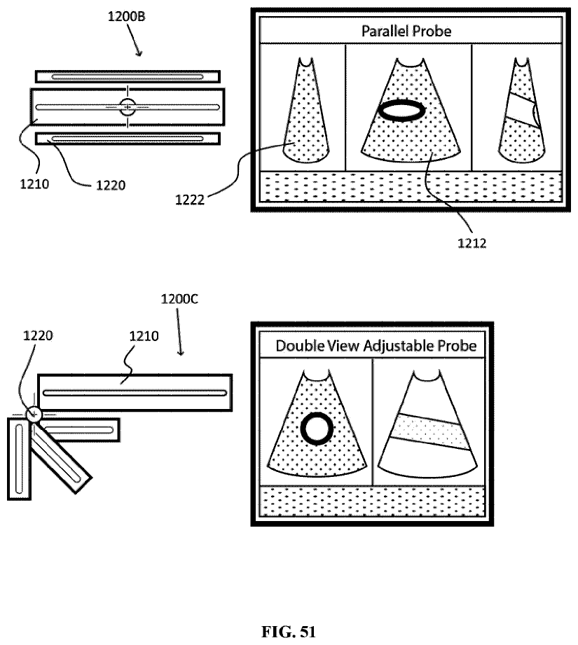

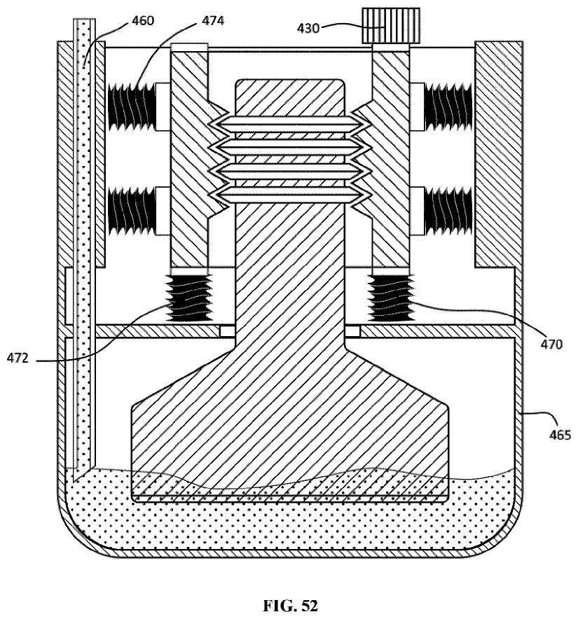

Referring now to FIG. 51 another embodiment of the multi-plane ultrasound device is illustrated. More specifically, a multi-plane ultrasound device 1200B is illustrated. The multi-plane ultrasound device 1200B comprises a primary transducer 1210 providing the first scanning plane 1212, and two secondary transducers 1220 positioned substantially parallel and on either side to the primary transducer 1210. Positioned in this manner, the secondary transducers 1220 may provide secondary scanning planes 1222 being parallel to the longitudinal axis of the targeted vein. Understandably, the secondary scanning planes 1222 would therefore display the needle when said needle is displaced beyond the first scanning plane 1212 thereby prompting the medical practitioner to readjust said needle.

Still referring to FIG. 51, yet another embodiment of the multi-plane ultrasound device is illustrated. More specifically, a multi-plane ultrasound device 1200C is presented comprising a primary transducer 1210 providing the first scanning plane 1212, and a pivoting secondary transducer 1220 positioned to pivot about a predetermined axis being about normal to the scanning surface. The pivoting secondary transducer 1220 may be pivoted to provide a secondary scanning plane 1222 at any desirable angle relative to the primary scanning plane 1212.

Understandably, the multi-plane ultrasound device 1200 may comprise any desirable number of transducers to detect a plurality of distinct scanning planes equal to the number of transducers. Preferably, the multi-plane ultrasound device 1200 comprises two transducers.

The multi-plane ultrasound device 1200 may further comprise a controller (not shown) configured to process the signals produced by the plurality of transducers 1200, such as the primary and secondary transducers 1210, 1220 and to output a video feed to a monitor of the said device 1200. In a preferred embodiment, the video feed produced by the controller displays the scanning planes produced by all the transducers, such as the primary and secondary transducers 1210, 1220 and provides to a medical practitioner a concurrent view of both scanning planes. As such, the controller may be configured to combine the feeds of each of the transducers 1210, 1220 into a single image output.

In certain embodiments, the multi-plane ultrasound device 1200 may further comprise an audio emitting device, such as a speaker (not shown) operable by the controller. In such embodiments, the controller may be further configured to activate a visual or auditory alarm when a needle is detected within a predetermined scanning plane. For example, an alarm may be activated when a needle is detected in a secondary scanning plane 1222 indicating that it has been displaced beyond the first scanning plane 1212 and is therefore in need of readjustment.

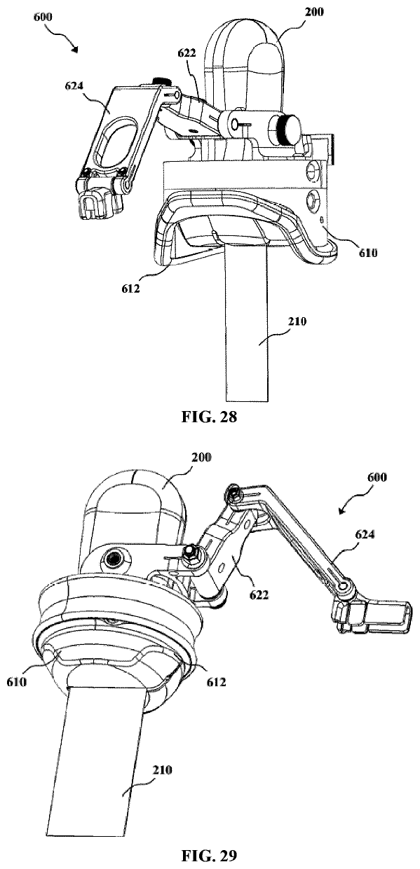

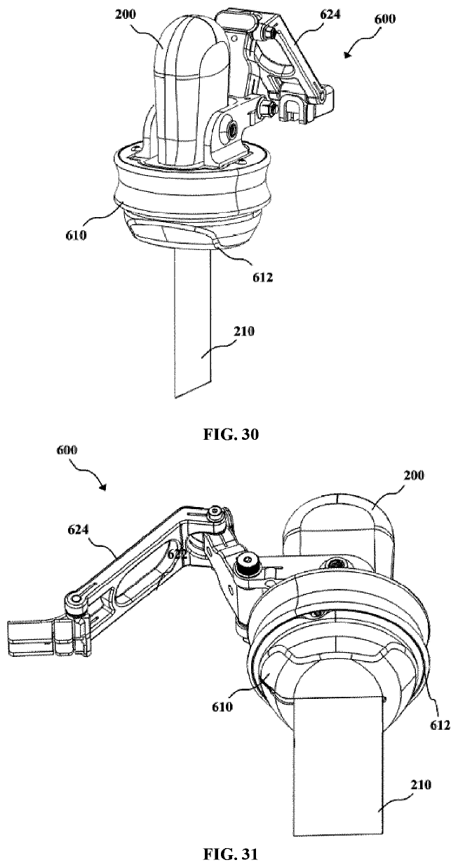

Referring now to FIGS. 26 to 31, another embodiment of an ultrasound transducer device 600 emitting a scanning plane 210 is illustrated. The device 600 comprises a lower support 610 and a pivoting arm 620 configured to receive the medical device 300, such as a catheter.

In certain embodiments, the lower support 610 may comprise a cross-sectional shape adapted to distribute pressure over a larger surface area onto a patient skin surface such as to maintain a desirable level of pressure on the patient's member under examination. In a preferred embodiment, the cross-sectional shape of the lower support 610 distribute pressure around the area that is scanned by the device 600. As such, the

The ultrasound transducer device 600 of the illustrated embodiment comprises a pivoting arm 620. The pivoting arm 620 comprises first and second members 622, 624 similarly configured to limit a motion of the medical device about a range of motion coplanar with the scanning plane 210. A first end of the first member 622 is pivotally attached to the top support of the device 600 about a single first axis. A second end of the first member 622 is pivotally attached to a first end of the second member 624 about a single second axis, the second axis being substantially parallel to the first axis. In certain embodiments, the first and second members 622, 624 may be telescopic to allow an extension along their longitudinal axis. The extension of the first and second members 622, 624 may provide a greater range of motion of the medical device 300 during a procedure. One or more of the first and second arms 622, 624 may be angled to define an alternative and/or more desirable range of motion of the medical device 300. In the embodiment illustrated in FIGS. 18 to 23, the second arm 624 is thus angled.

The pivoting arm 620 further comprises a third member 626 pivotally attached to the second member about a third axis, the said third axis being substantially parallel to the first and second axis. The third member 626 is adapted to receive the medical device to be inserted in a body portion of the subject.

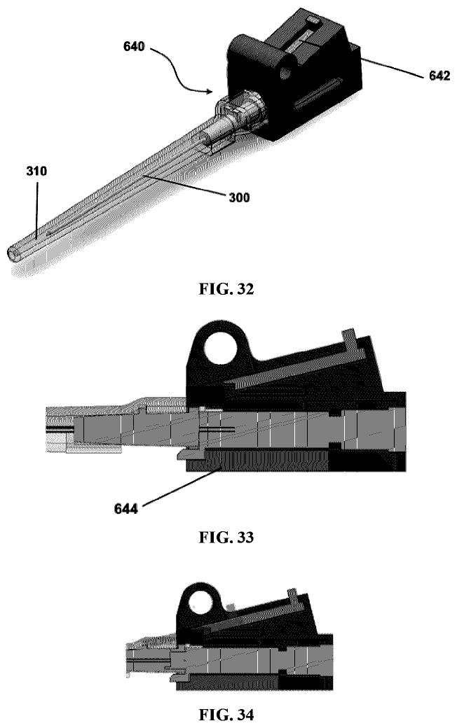

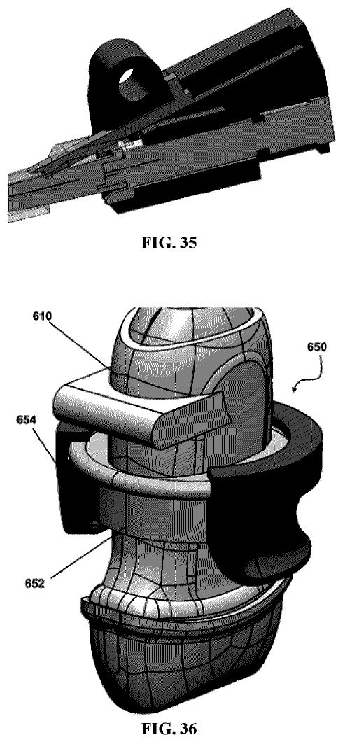

Referring now to FIGS. 32 to 35, an embodiment of a medical device attaching assembly 640 is illustrated. The medical device attaching assembly 640 generally allows mounting the medical device yet detaching it while limiting the movement of the said medical device when inserted in the body of the subject. The medical device attaching assembly may further allow detaching and removing a cover 310 over the medical device 300 without touching the said cover 310. Not touching the cover 310 generally aims at maintaining a sanitized environment and maintaining stability of the position of the device 600.

In the illustrated embodiment, the attaching assembly 640 comprises a slideable member 642 adapted to move toward and away of a portion of the cover 310. As such, when pushed against the cover, the slideable member 642 pushes the cover 310 away from the medical device 300. In some embodiments, the slideable member 642 is resiliently attached to the assembly 640 to return to the initial position after being pushed.

In the illustrated embodiment, the attaching assembly 640 further comprises a chariot or push member 644 adapted to move toward and away of the medical device 300.

In use, a user positions the device 600 over a zone where to insert the medical device and position the medical device 300 using the pivoting arm 620. Once positioned, the user first pushes on the slideable member 642 to remove the cover without touching it. Next, the user moves the chariot 644 or triggers its movement to release the medical device 300 from the attaching assembly 640.

Still referring to FIGS. 26 to 31, the support 610 may further comprise a curved edge 612 defining a curvature profile being mirrored about the scanning plane 210. To that end, the curved edge 612 may allow a pivoting of the ultrasound transducer mount 600 about an axis being normal to the scanning plane 210. The pivoting of the ultrasound transducer mount 600 may provide a medical practitioner a desirable increase in the range of motion of the medical device 300 during a procedure thereby providing a better view of the needle within the scanning plane 210.

In certain embodiments, the ultrasound transducer mount 600 may comprise one or more actuators (not shown) configured to pivot or rotate the ultrasound transducer 200 within the ultrasound transducer mount 600 to similarly provide a better view of the needle within the scanning plane 210.

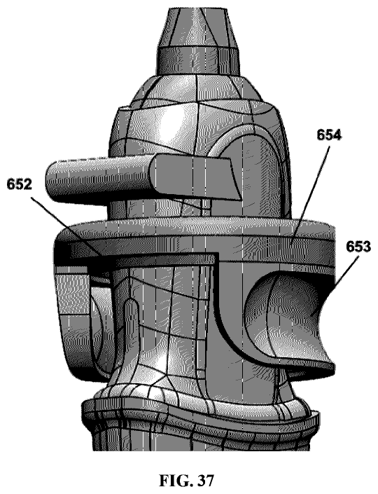

Referring now to FIGS. 36 and 37, an embodiment of a rotation assembly 650 is illustrated. The rotation assembly 650 generally aims at rotating the support member 610 of the device 600. In use, a first hand of the user holds the rotation assembly 650 and another hand of the user manipulates the device 600, such as through the body 610 or the articulated arm 620. The rotation assembly 650 allows rotation of the device around a substantially vertical axis.

In the illustrated embodiment, the rotation assembly 650 comprises an attachment member 652 mounted to the body 610 of the device 600. In such an embodiment, the attachment member 652 surrounds the body 610. The rotation assembly 650 further comprises a disk or rotation member 654 adapted to rotate or slide over the attachment member 652. As such, the disk 654 may freely move around the body 610 of the device 600. In some embodiments, the rotation member 654 may further comprise a handle or manipulable portion 653. The handle 653 is typically designed to be ergonomic to a hand of the user.

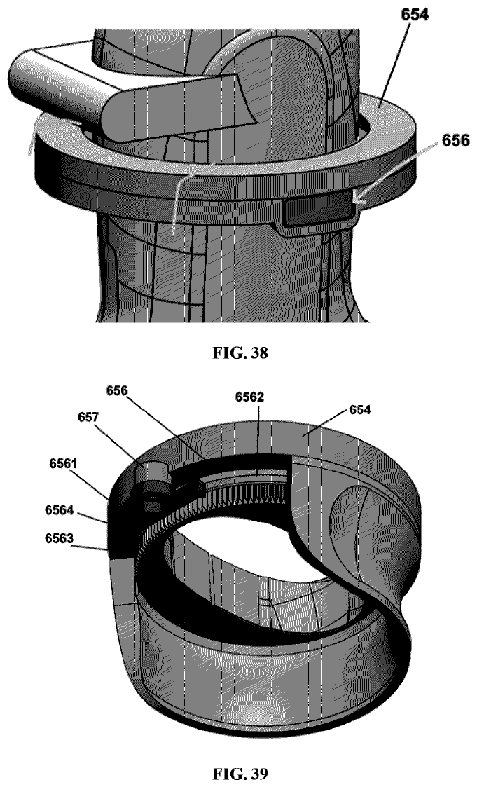

Referring now to FIG. 38, an embodiment of the rotation assembly 650 comprising a brake or locking mechanism 656 is shown. The locking mechanism 656 is typically adapted to lock the rotation member 654 in place by default. When a user presses or activates the locking, the rotation member 654 freely moves over the attachment member 652. Understandably, any known mean or mechanism adapted to limit or lock the rotation of the rotation assembly in relation to the body may be used within the scope of the present invention.

Referring now to FIGS. 39 to 42, another embodiment of a brake or locking mechanism 656 is illustrated. The brake mechanism 656 comprises a pivoting point 657 attached to the rotation member 654. The brake mechanism 656 further comprises a disk 6562 attached to the body 610 of the device 600. The disk 6562 comprises teeth or engagement members 6563. The engagement members 6563 are typically positioned on the outskirt or periphery of the disk 6562. The brake mechanism 656 further comprises a locking member 6561 pivotally attached to the pivoting point 657. The locking member 6561 typically comprises one or more engagement member 6564 adapted to interlock or mate with the engagement members 6563 when the locking member 6561 is pivoted on one side toward the said disk 6562. In use, a user pushes the locking member 6561 one side to lock the disk when the two engagement members 6563 and 6564 lock together and to unlock the rotation movement when the locking member 6561 is moved back in an initial position.

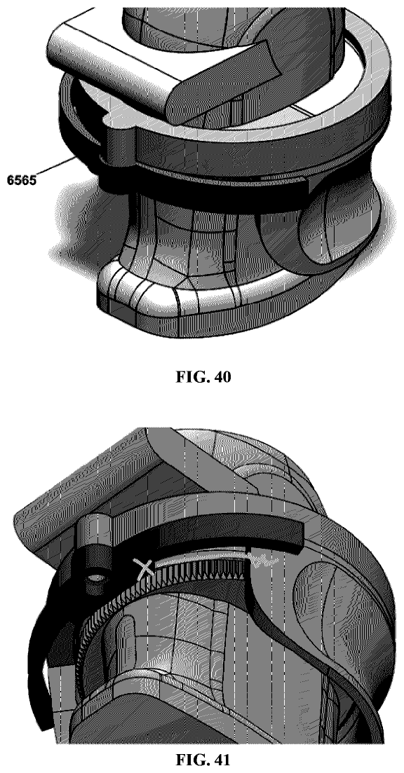

Referring now to FIGS. 40 and 41, In some embodiments, the brake mechanism 656 may further comprise a handle 6565 connected to the locking member 6561 to facilitate the operation of the brake mechanism 656.

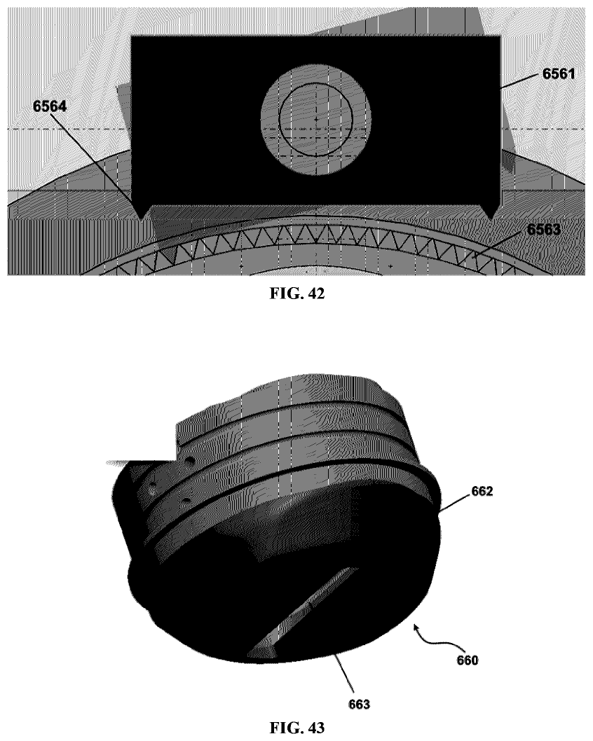

Referring now to FIG. 42, an embodiment of a locking member 6561 is illustrated in a locked (purple) and unlocked position (blue).

Referring back to FIG. 36, in yet other embodiments, the attachment member 652 may be vertically slidable over the body 610 of the device 600. As such, the rotation assembly 650 may be vertically positioned to improve ergonomics of the device for different users. In such an embodiment, the attachment member 652 may comprise a slider adapted to surrounds yet vertically move around the body 610.

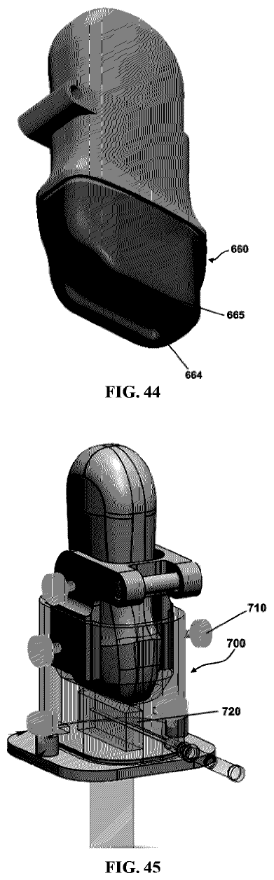

Referring now to FIGS. 43 and 44, a gel chamber 660 covering the lower portion of the body 610 of the device 600 is shown. The gel chamber 600 generally comprises an outer membrane 662 adapted to create an inner chamber (not shown) between the lower portion of the body 610 and the said inner portion of the outer membrane 662. The membrane 662 comprises a slot or aperture 663 to let the gel out of the chamber in operation. The chamber 660 is typically clipped or detachably attached to the body 610. By being detachably attached to the body 610, the chamber or the device 600 may be easily cleaned.

Referring to FIG. 44, in some embodiments, the chamber 660 comprises outer walls 665 adapted to elevate a portion of the bottom of the device 600. Such elevation generally aims at reducing the creation of bubbles of air and to let gel flows between the said wall. Furthermore, the walls allow having pressure around the area of the body being scanned by the device 600.

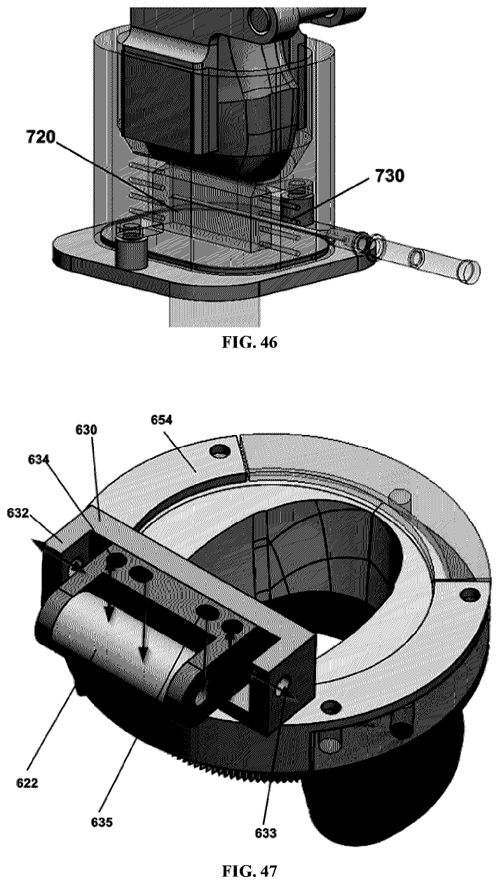

Referring now to FIGS. 45 and 46, embodiments of a mechanism to align or calibrate the device 600 are illustrated. In such embodiment, the calibration device 700 comprises a plurality of holding points 710 to attach the calibration device 700 to the body 610 of the transducer 600. The holding points 710 may be embodied as screws with rubber tips, as illustrated. Referring to FIG. 32, the calibration device 700 comprises an inner container 720. The inner container 720 may be filled with a liquid, such as water, to calibrate the direction of the medical device 300, such as a catheter. The transducer 600 detects the liquid in the container and a user may then calibrate or align the arm to let the transducer 600 scan the medical device 300 within the container.

Referring now to FIG. 46, another embodiment of the calibration device 700 is shown. In such an embodiment, the calibration device 700 comprises one or more passages 730 adapted for the medical device 300 to pass through. The different passages ensure that the medical device 300 is adjusted to be insert at a predetermined depth. As illustrated, the calibration device 700 may comprise a plurality of passages 730 positioned at predetermined depths in the container 720.

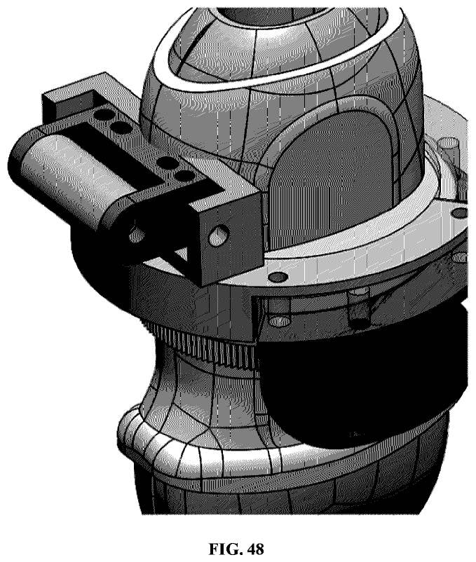

Referring now to FIGS. 47 and 48, the body 610 may comprise an arm attachment member 630. The attachment member 630 comprises an arm connecting member 632 mounted to the arm attachment member 630 using any type of attachments, such as fasteners. In the illustrated embodiment, the arm attachment member 630 comprises one or more apertures 633 adapted to receive a fastener and the arm connecting member 632 further comprises one or more apertures 634 and 635 also adapted to receive a fastener. In such an example, the fasteners when inserted in the apertures 633, 634 and 635 mount the arm 622 to the body 610. To calibrate the device, the user tightens or loosens the fasteners in the side apertures 633 to correct move the arm to the sides. The user further tights or loosens the top fasteners 634 to correct the height of the arm 620. The user may also tights or loosens other top fasteners 635 to correct the angle of the arm 620. Understandably, the calibration fasteners 633, 634 and 635 are illustrated as example of how to change the calibration of the arm 620. Any other known method to calibrate the arm 620 may be used within the scope of the present invention.

In yet other embodiments, the arm 620 could be mounted to the rotation member 654 thus pivoting around the device 600. In certain embodiments, the ultrasound transducer mount 600 may further comprise an orifice 660 adapted to receive a needle cap of the medical device 300. In particular, the orifice 660 may be configured to allow an insertion of the needle cap while thereafter retaining said needle cap therein. The orifice 660 may thus allow a medical practitioner to arrange a needle or catheter for insertion without requiring a manipulation of the needle, catheter and/or needle cap at any stage of the procedure.

The present invention is further directed to an ultrasound device 200 comprising a monitor (not shown). The monitor may be affixed to an upper end of the ultrasound device 200 and be configured to rotate about a transversal axis of the ultrasound device 200 and to tilt about a radial axis of the ultrasound device 200. The monitor may provide the medical practitioner a view of the captured ultrasound image during use of said ultrasound device 200.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. An apparatus to guide movement of a medical device insertable in a physical body, the apparatus comprising:
a mounting assembly for receiving an ultrasound transducer, the mounting assembly rotatable around the ultrasound transducer; and
an articulated arm having a first end attachable to the mounting assembly and a second end attachable to the medical device, the articulated arm moving along a single plane to position a longitudinal axis of the medical device in-plane.

2. The apparatus of claim 1, the articulated arm being used to rotate the mounting assembly.

3. The apparatus of claim 1, the mounting assembly comprising a pivoting base for receiving the ultrasound transducer and a rotation mechanism engaging rotation of the pivoting base.

4. The apparatus of claim 3, the pivoting base having a shape substantially matching a shape of a bottom portion of the ultrasound transducer.

5. The apparatus of claim 3, the pivoting base comprising a suspending mechanism adapted to receive a bottom portion of the ultrasound transducer.

6. The apparatus of claim 3, the pivoting base comprising a liquid fillable sealed chamber between a bottom portion of the ultrasound transducer and the physical body.

7. The apparatus of claim 3, the pivoting base being shaped to lock with a bottom portion of the ultrasound transducer.

8. The apparatus of claim 3, the pivoting base comprising a brake to maintain the pivoted base to a specific angle.

9. The apparatus of claim 1, the second end of the articulated arm being selectively attachable and detachable from the medical device.

10. The apparatus of claim 1, the second end of the articulated arm comprising a medical device attaching assembly for mounting yet unmounting the medical device using one hand.

11. The apparatus of claim 10, the medical device attaching assembly comprising a touchless cover removal mechanism to remove a cover of the medical device.

12. The apparatus of claim 11, the touchless cover removal mechanism comprising a slidable member to move the cover of the medical device away from the medical device.

13. (canceled)

14. The apparatus of claim 10, the medical device attaching assembly being a slideable chariot.

15. The apparatus of claim 10, the medical device attaching assembly comprising a lateral force absorption member.

16. The apparatus of claim 15, the lateral force absorption member being resilient along a longitudinal axis of the medical device.

17. The apparatus of claim 1, the second end of the articulated arm comprising a slidable member to move the medical device toward and away of the mounting assembly.

18. The apparatus of claim 10, the medical device attaching assembly conforming with an external surface of the medical device.

19-25. (canceled)

26. An assembly for attaching yet detaching a medical device insertable in a physical body to an ultrasound transducer using a single hand, the assembly comprising:
a member for releasably attaching the medical device to the assembly to be in-plane;
a member for pivotably attaching the assembly to an elongated arm of the ultrasound transducer.

27. The assembly of claim 26 further comprising a touchless cover removal mechanism to remove a cover of the insertable medical device.

28. (canceled)

29. (canceled)

30. The assembly of claim 26 further comprising a lateral force absorption member.

31. (canceled)
